(19) United States
(12) Patent Application Publication
(10) Pub. No.: US 2016/0228006 A1
(43) Pub. Date: Aug. 11, 2016

(54) SYSTEM, METHOD AND COMPUTER-ACCESSIBLE MEDIUM FOR UTILIZING DISCRETE FOURIER-TRANSFORM FOR FREQUENCY NEAR-INFRARED SPECTROSCOPY

(71) Applicant: THE GENERAL HOSPITAL CORPORATION, Boston, MA (US)

(72) Inventors: BERHARD ZIMMERMANN, Cambridge, MA (US); DAVID A. BOAS, Winchester, MA (US)

(21) Appl. No.: 15/026,727

(22) PCT Filed: Oct. 1, 2014
(86) PCT No.: PCT/US14/58664

§ 371 Date: Apr. 1, 2016

Related U.S. Application Data
(60) Provisional application No. 61/885,131, filed on Oct. 1, 2013.

(51) Int. Cl. A61B 5/00; A61B 6/00; G01N 21/359; A61B 6/02

(52) U.S. Cl. CPC A61B 5/0059; A61B 6/025; A61B 6/502; A61B 5/7257; G01N 21/359; G01N 2201/0612; G01N 2201/0833

(57) ABSTRACT

An exemplary system, apparatus, method and computer-accessible medium for determining information regarding a sample(s), can be provided, which can include, for example, a source arrangements(s) which can provide a first radiation(s), whose intensity can vary over time, to the sample(s), a detector arrangement(s) which can be configured to receive a second radiation(s) from the sample(s) based on the first radiation(s) and a computer arrangement(s) which can be configured to simultaneously determine the information regarding the sample(s) at a plurality of frequencies of the second radiation(s).

### CROSS-REFERENCE TO RELATED APPLICATIONS

This application relates to and claims priority from U.S. Patent Application No. 61/885,131, filed on Oct. 1, 2013, the entire disclosure of which is incorporated herein by reference.

### FIELD OF THE DISCLOSURE

The present disclosure relates generally to near-infrared spectroscopy, and more specifically, to exemplary embodiments of exemplary system, method and computer-accessible medium for utilizing a discrete Fourier-transform for frequency near-infrared spectroscopy.

### BACKGROUND INFORMATION

Frequency domain (“FD”) near-infrared spectroscopy (“NIRS”) is a well-established procedure for measuring tissue optical properties. In this procedure, e.g., an oximeter transmits a radio frequency (“RF”) intensity modulated light, at near-infrared wavelengths, through human tissue. By measuring amplitude and phase of the transmitted light, it may be possible to calculate absolute absorption and scattering coefficients of the tissue, which can facilitate the calculation of absolute concentrations of oxy- and deoxyhemoglobin. (See, e.g., References 1 and 2).

One application of FD-NIRS is a diffuse optical tomography (“DOT”) of the breast. A FD-NIRS system for this application has been previously used, and utilizes the homodyne in-phase and quadrature demodulation technique. (See, e.g., References 4 and 5). This FD-NIRS system has been successfully used in conjunction with a continuous wave (“CW”) NIRS imager and co-registration of tomographic X-ray scans for imaging breast tumors. (See, e.g., Reference 6). It has been recently shown that additional information can be obtained by analyzing dynamic changes in optical tissue parameters induced by compression, such as the one exerted by the mammography system. (See, e.g., References 7 and 8). A motivation to build a second generation system can be therefore to increase the temporal resolution of the RF imaging system, which can be combined with X-ray mammography, e.g., to probe compression induced hemodynamics.

Thus, it may be beneficial to provide exemplary system, method and computer-accessible medium for utilizing a discrete Fourier-transform for frequency near-infrared spectroscopy, which can overcome at least some of the deficiencies described herein above.

### SUMMARY OF EXEMPLARY EMBODIMENTS

An exemplary system, apparatus, method and computer-accessible medium for determining information regarding a sample(s), can be provided, which can include, for example, a source arrangements(s) which can provide a first radiation(s), whose intensity can vary over time, to the sample(s), a detector arrangement(s) which can be configured to receive a second radiation(s) from the sample(s) based on the first radiation(s) and a computer arrangement(s) which can be configured to simultaneously determine the information regarding the sample(s) at a plurality of frequencies of the second radiation(s).

In some exemplary embodiments of the present disclosure the first radiation(s) or the second radiation(s) can include an optical radiation. The frequencies of the second radiation(s) can be intensity modulated frequencies. A frequency modulating arrangement can be configured to cause the first radiation(s) to be intensity modulated to provide a modulated first radiation to the sample(s). The frequencies of the second radiation(s) can be related to further frequencies of the intensity modulated first radiation. The detector arrangement(s) can include a demodulation arrangement which can be configured to receive and simultaneously demodulate the second radiation(s) at the frequencies to generate demodulated information. The information regarding the sample(s) can be determined based on the demodulated information. The demodulation arrangement can include a digitizing arrangement which can be configured to receive and digitize the second radiation(s) prior to the demodulation thereof.

In certain exemplary embodiments of the present disclosure, the demodulation arrangement can include a hardware adder circuitry which can be configured to demodulate the second radiation(s) without a hardware multiplier arrangement. A frequency modulating arrangement can be configured to cause the first radiation(s) to be intensity modulated to provide a modulated first radiation to the sample(s), wherein the frequency modulating arrangement can be further configured to modulate further frequencies of the first radiation(s) based on a sampling rate(s) provided by the digitizing arrangement. The demodulation arrangement can include a hardware adder circuitry(s) which can be configured to provide modified information regarding the second radiation(s), and the information regarding the sample(s) can be determined based on the modified information.

In some exemplary embodiments of the present disclosure, the information regarding the sample can be determined based on the modified information to achieve a Discrete Fourier Transform result of the second radiation(s). At least two bins can be individually computed based on the second radiation(s) using the DFT and a Goertzel procedure. The source arrangement(s) can include at least two radiation generating arrangements configured to generate at least two further radiations, and a radiation combining arrangement can be configured to combine the two further radiations into a third radiation(s) that can be associated with the first radiation(s). The radiation generating arrangements can include lasers. The radiation combining arrangement can include a dichroic mirror. A galvanometer(s) can be configured to receive the third radiation(s) and generate a fourth radiation(s). A microcontroller can be configured to adjust the galvanometer(s) to a particular angle.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary image of an exemplary tomosynthesis system according to an exemplary embodiment of the present disclosure;

FIG. 1B is an exemplary image of an exemplary frequency domain near-infrared spectroscopy system according to an exemplary embodiment of the present disclosure;

FIG. 2 is an exemplary schematic diagram of an exemplary frequency domain near-infrared spectroscopy system according to an exemplary embodiment of the present disclosure;

FIG. 3A is an exemplary image of an exemplary source arrangement including a clock generator, laser drivers, lasers and a Galvo, according to an exemplary embodiment of the present disclosure;

FIG. 3B is an exemplary image of an exemplary detector card according to an exemplary embodiment of the present disclosure;

FIG. 4 is an exemplary diagram illustrating a flow of Goertzel's procedure according to an exemplary embodiment of the present disclosure;

FIG. 5 is an exemplary diagram illustrating the flow of Goertzel's procedure with additional recursive loops according to another exemplary embodiment of the present disclosure;

FIG. 6 is an exemplary diagram illustrating the flow exemplary system, method and computer-accessible medium according to still another exemplary embodiment of the present disclosure;

FIG. 7 is an exemplary diagram illustrating the exemplary filter structure and flow for a first modulation frequency according to a further exemplary embodiment of the present disclosure;

FIG. 8A is an exemplary graph illustrating signal magnitude versus incident optical power generated using exemplary system, method and computer-accessible medium according to an exemplary embodiment of the present disclosure;

FIG. 8B is an exemplary graph illustrating signal phase versus incident optical power generated using exemplary system, method and computer-accessible medium according to an exemplary embodiment of the present disclosure;

FIG. 9 is an exemplary flow diagram illustrating an exemplary method for determining information regarding a sample according to an exemplary embodiment of the present disclosure; and

FIG. 10 is an illustration of an exemplary block diagram of an exemplary system in accordance with certain exemplary embodiments of the present disclosure.

Throughout the drawings, the same reference numerals and characters, unless otherwise stated, can be used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures.

### DETAILED DESCRIPTION OF EXEMPLARY EMBODIMENTS

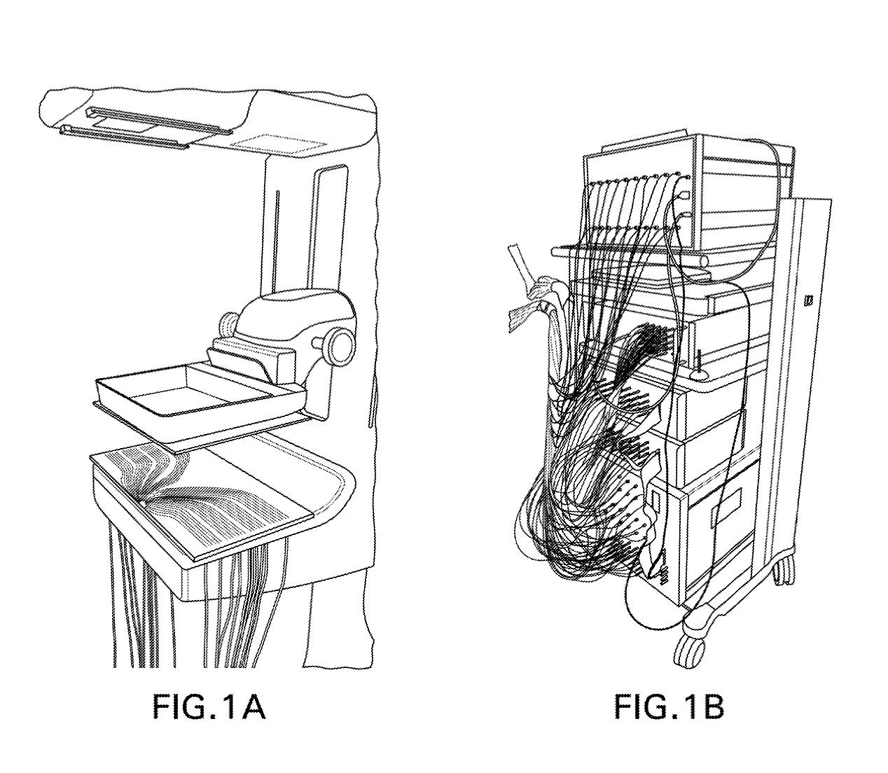

An exemplary tomographic breast imaging system (e.g., “TOBI2”) according to an exemplary embodiment of the present disclosure is shown in FIGS. 1A and 1B. The TOBI2 includes four components. An exemplary digital tomosynthesis instrument can perform a high-resolution limited-angle x-ray tomography on the inserted breast. An exemplary x-ray translucent optical probe can be attached to the compression paddles of the tomosynthesis machine. Upper paddle can house the optical source fibers, the lower paddle can house the detector fiber bundles, and light can be sent from the CW-NIRS and FD-NIRS instruments through optical fibers to the source plate. It can then pass through the breast tissue and can be collected by the detector fiber bundles on the other side. The transmitted light can be provided to avalanche photo diode (“APD”) detectors in the CW-NIRS and FD-NIRS systems. The FD-NIRS system measure both amplitude and phase of the transmitted light, the CW-NIRS may only measure the amplitude.

### Exemplary Hardware

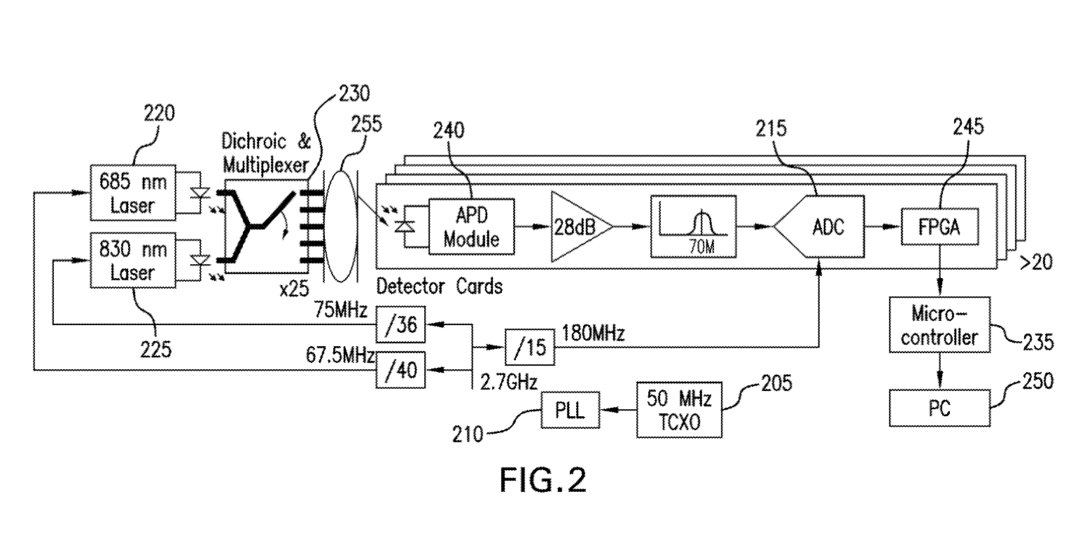

A schematic diagram of the exemplary FD-NIRS system according to an exemplary embodiment of the present disclosure is shown in FIG. 2. For example, a 50 MHz reference clock 205 signal can be generated by a temperature compensated crystal oscillator (“TCXO”). A 2700 MHz clock signal can be derived from this reference by a phase locked loop (“PLL”) 210. The laser modulation frequencies, as well as the sampling clock for the analog-to-digital converters (“ADC”) 215, can then be derived from PLL 210 output by an integer division. A division by two for the laser modulation frequencies may be done on the laser driver boards to keep the signals as localized as possible to simplify shielding of electro-magnetic interference (“EMI”). A distribution of the clock signal to, e.g., many or all 20 detector cards can be accomplished by two low voltage differential signaling (“LVDS”) dock fanout buffers. A clock jitter can also be minimized.

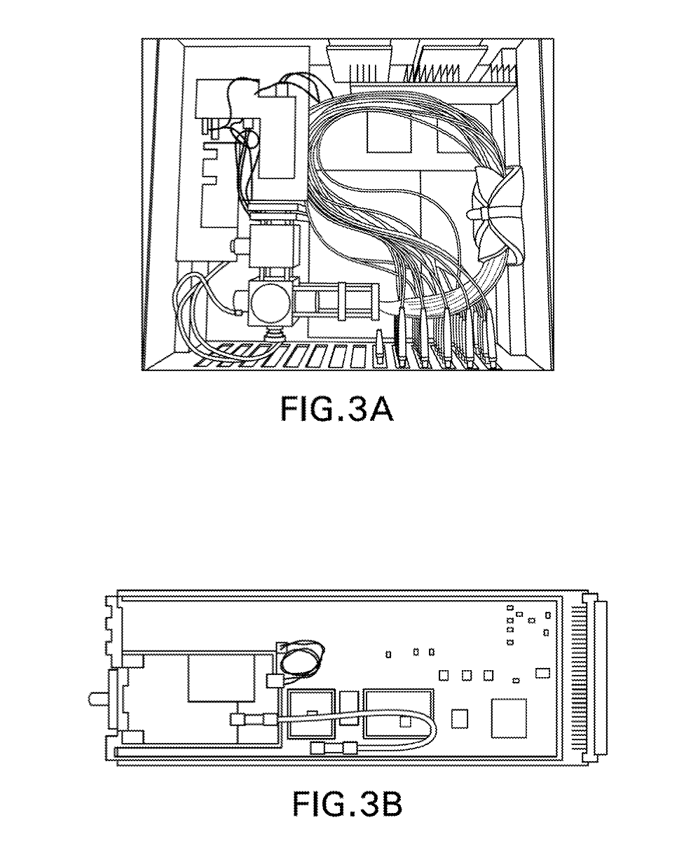

Exemplary light sources can include, e.g., a 685 nm laser diode 220 modulated at 67.5 MHz and a 50 mW 830 nm laser diode 225 modulated at 75 MHz. Beams of both diodes can be combined by a radiation combining arrangement 230 (e.g., being or including a dichroic mirror, a filter, a beam splitter etc.), and can then be launched via a 2D scanning galvanometer 255 (e.g., Thorlabs GVS002) into 200 μm multimode silica fibers. For example, about 25 fibers can be arranged in an array so that the illuminated fiber can be selected by steering the galvanometer to the appropriate exemplary angle. Additionally the beam can be steered into a beam dump, which can facilitate the system to acquire the dark signal. Using a standard galvanometer as an optical multiplexer can be a very cost effective solution, and can facilitate rapid changes of the selected source location. The exemplary galvanometer can also facilitate the increase in the number of source positions. Such galvanometer can be controlled by its own dedicated microcontroller 235 (e.g., Atmel ATmega2560) to ensure or otherwise facilitate fast switching between fibers. The galvanometer can also contain position calibration procedures. An image of the exemplary arrangement described herein is shown in FIGS. 3A and 3B, which illustrate a source box (e.g., arrangement) that contains a clock generator, laser drivers and a Galvo for optical multiplexing (see FIG. 3A), and a detector card (See FIG. 3B).

After light (or other electro-magnetic radiation) passes through tissue, it can be collected by silica fiber bundles and routed to the photo detectors. Each of the 20 detection channels (which can be more or less than 20) can be provided on its own printed circuit board (see FIGS. 3A and 3B) to facilitate easy replacement and expandability. On each of the detector boards, an avalanche photo diode (“APD”) module 240 (e.g., Hamamatsu C5331-04) can detect the optical signal. The exemplary module can also include a first gain stage in form of a low noise transimpedance amplifier, as well as a high voltage supply to bias APD 240. The bias voltage can be temperature-compensated to keep the gain of APD 240 at least substantially constant.

Following the APD 240 module, the signal can be further amplified by a high speed, low noise current feedback op-amp. Then, the signal can be filtered with, e.g., a 63 to 77 MHz band-pass in order to reject other signals, especially the light coming from the CW-NIRS instrument, which could otherwise saturate the analog to digital converter. The single ended signal can be converted into a differential signal by a transformer. Further, the signal can be fed into a differential amplifier which can serve as the last gain stage, and also as an ADC buffer and an anti-aliasing low-pass filter. Each section of the analog signal chain can be individually shielded to prevent inter channel cross talk between neighboring detector cards.

The signal can be sampled at, e.g., about 180 mega samples per second (“MSPS”) and at, e.g., about a 16 bit resolution by a high speed analog to digital converter (e.g., Linear Technologies LT2209). ADC 214 can be directly connected to a low-cost field-programmable gate array “FPGA” 245 (e.g., FPGA, Xilinx Spartan6 LX9) which can demodulate the signal.

Most or all of the detector cards, as well as the control card, can be connected by a common backplane. A microcontroller 235 (e.g., Atmel ATmega2S60) can collect the data from all detectors cards via a serial peripheral Interface bus (“SPI”) with LVDS. The data can then be sent on to the PC 250 via USB for further signal processing and data recording.

### Exemplary Demodulation Procedure

As discussed herein, the analog signal can be sampled at about 180 million times per second at a 16 bit resolution. From this raw data stream, the signals from the 690 nm and 830 nm lasers can be extracted. In a standard exemplary analog instrument, this can be accomplished using, e.g., a homo detection or a heterodyne detection with a mixer for down conversion and then a slow ADC for sampling the in-phase and quadrature signals.

In the exemplary system, method and computer-accessible medium according to an exemplary embodiment of the present disclosure, this can be done digitally. For example, a Fast Fourier Transform (“FFT”) procedure can be used. However, a fairly large amount of frequency bins can be utilized to keep the width of each individual bin small enough, and thus, the memory and computational needs can be very high, and a cost effective solution for a 20 channel instrument may not be possible. The exemplary FFT procedure can compute or otherwise determine, e.g., using a computer or a microcontroller, most or all N bins simultaneously, while the exemplary system, method and computer-accessible medium, according to an exemplary embodiment of the present disclosure, may only need to obtain the values from two (or more) bins. Therefore, a Discrete Fourier Transform (“DFT”) can be implemented according to an exemplary embodiment of the present disclosure, and the interesting bins (e.g., two or more) can be individually computed, without having to compute all the other bins.

The standard DFT X[k] can be computed as shown in Eq. (1), where x[n] can be the input signal,

$${W}_{N} = {e}^{- J ( \frac{2 π}{N} )}$$

### and K

X[K]=Σᵣ₌₀^{N-1}x[r]W_{N}^{kr} (1)

As can be seen herein, a direct implementation of this equation in a FPGA may not be easy. The exemplary procedure according to an exemplary embodiment of the present disclosure can utilize complex multiplications, and all the complex factors W_{N}^{kr} have to be computed on the fly or stored in a lookup table.

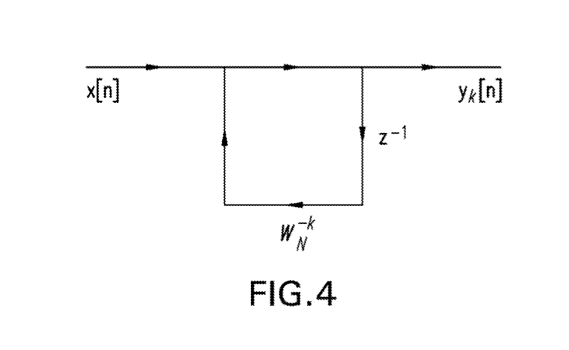

In order to simplify this, an exemplary Goertzel procedure can be used. For example, a specific bin k of the N-point DFT can be computed by feeding the signal into a system with impulse response W_{N}^{-kn}u[n], which can be initially at rest. (See, e.g., Reference 9). u[n] can be the unit step function. The desired result can then be the Nth output value (see Eq. (2) and Eq. (3) herein) of the exemplary system, method and computer-accessible medium according to an exemplary embodiment of the present disclosure. (See also exemplary flow illustrated in FIG. 4).

X[K]=yₖn]N (2)

yₖ[n]=W_{N}^{-k}yₖ[n−1]+x[n]

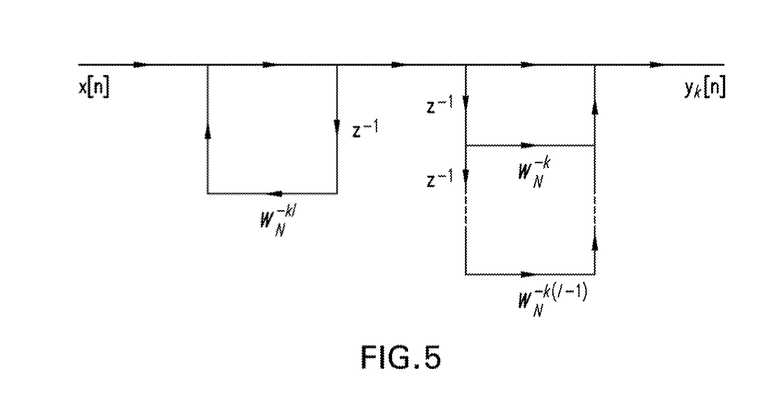

The resulting exemplary procedure can be better suited for implementation in an FPGA; the multiplication factor can be constant, and the complete input sequence does not need to be kept in memory. The exemplary procedure can utilize one complex multiplication, or 4 real multiplications, per input sample. Further, the utilized adder and multiplier in the recursive loop may only have a combined latency of one clock cycle, which may not be possible to implement in low cost FPGA's at the numerical resolution and speed. To overcome this problem with the insufficient latency in the recursive loop, the one sample delay element z⁻¹ can be changed to an l sample delay element z⁻¹. The flow changes are shown in the graph of FIG. 5. Note that some non-recursive elements have been added to compensate for the added delay and keep the overall transfer function unchanged.

The exemplary transfer function of Eq. (4) is shown in FIG. 5 for the case when l=2. The structure can be further simplified by multiplying the numerator and denominator with a common factor as shown in Eq. (5) and Eq. (6). (See e.g., Reference 9).

$$\begin{matrix}{H}_{k} ( z ) = \frac{1 + {z}^{- 1} {W}_{N}^{- k}}{1 - {z}^{- 2} {W}_{N}^{- 2 k}} & ( 4 ) \\ {H}_{k} ( z ) = \frac{( 1 + {z}^{- 1} {W}_{N}^{- k} ) ( 1 - {z}^{- 2} {W}_{N}^{2 k} )}{( 1 - {z}^{- 2} {W}_{N}^{- 2 k} ) ( 1 - {z}^{- 2} {W}_{N}^{2 k} )} & ( 5 ) \\ {H}_{k} ( z ) = \frac{1 + {z}^{- 1} {W}_{N}^{- k} - {z}^{- 2} {W}_{N}^{2 k} - {z}^{- 3} {W}_{N}^{k}}{1 - 2 cos ( \frac{2 π}{N} 2 k ) {z}^{- 2} + {z}^{- 4}} & ( 6 )\end{matrix}$$

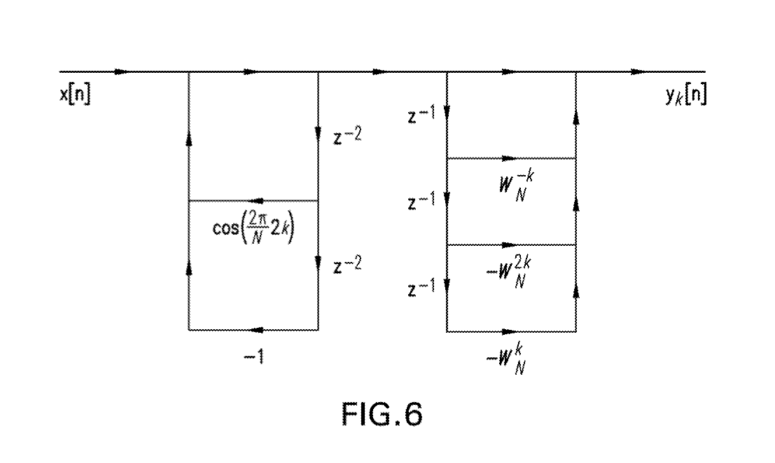

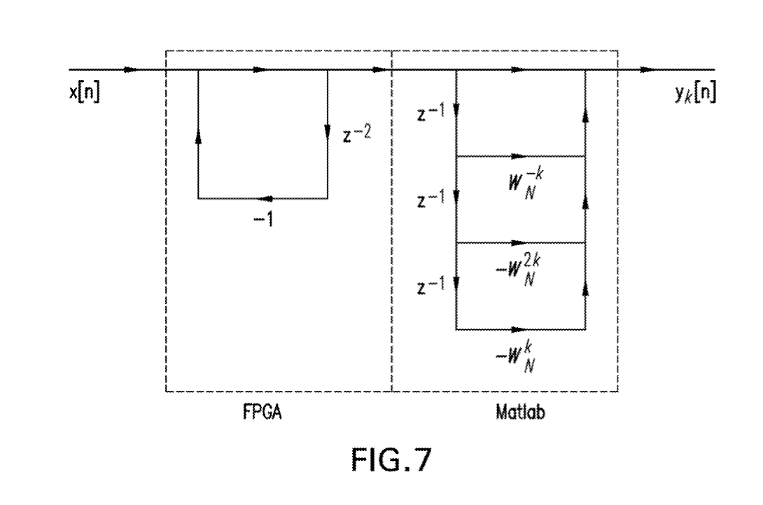

A complex multiplication in the recursive loop can be replaced with a real multiplication, at the expense of an additional term in the non-recursive part. The modulation frequencies of the instrument can be chosen within a particular range. Thus, if the modulation frequency of one laser can be chosen to be ⅜*180 MHz=67.5 MHz, corresponding to k=⅜*N, the cosine can become zero, and the structure can be further simplified as shown in FIGS. 6 and 7. The recursive loop can be reduced to a simple adder; for a real input sequence x[n] the numbers can stay real in this part of the computation. A similar structure can be found for the case where l=3, and the modulation frequency can be about 75 MHz.

The use of the exemplary system, method and computer-accessible medium according to an exemplary embodiment of the present disclosure can result in minimal resource requirements needed by the FPGA. For example, for a 4 million point DFT computation, only one 36 bit adder would be needed, and the adder can have a latency of up to four clock cycles, which can facilitate the use of pipelining in the exemplary implementation. The complex multiplications in the non-recursive structure can all be done in, for example, Matlab, since only the last four results of the recursive structure can be needed, and the amount of data to be transferred can be fairly low.

The exemplary system, method, and computer-accessible medium according to an exemplary embodiment of the present disclosure can be implemented on the FPGA of each detector card. For each modulation frequency, two OFT's with 50% overlap and length of about N=: 4·10⁶ can be calculated in real time. This can result in an output data rate of, e.g., about 90 Hz. None of the about 180 million samples per second that the ADC can acquire can be used for calculation of the output data. For this exemplary implementation, only four 36 bit adders, and some control logic, can be needed. Everything fits even in the smallest FPGA model of Xllinx's low cost Spartan 6 line, with room to add additional frequencies if needed later on.

### Exemplary Results

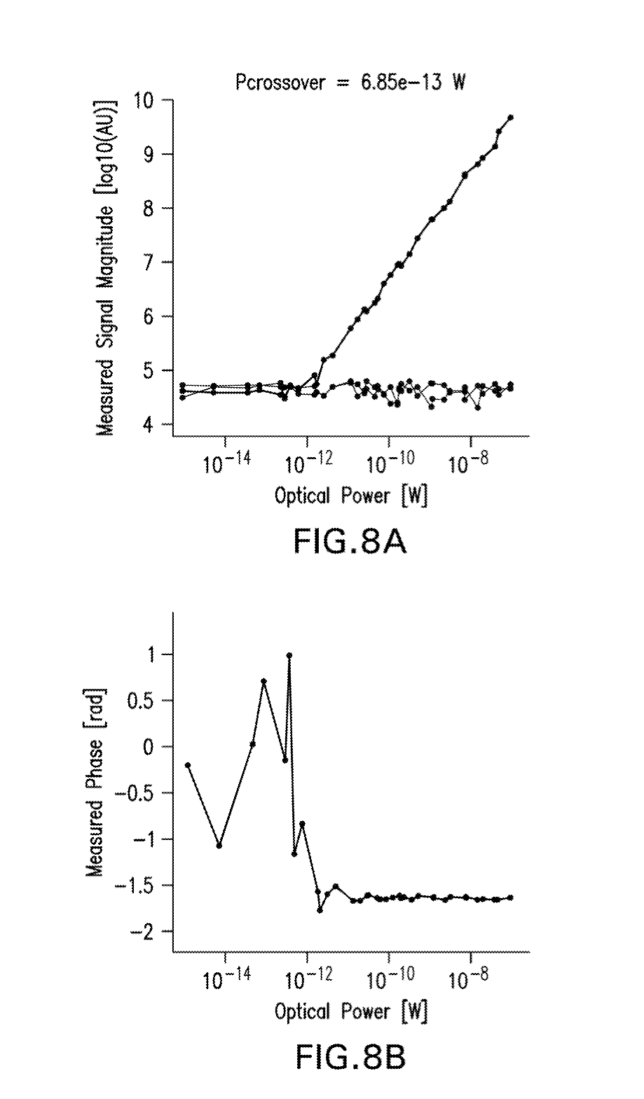

FIG. 8A illustrates a graph of of signal magnitude vs. incident optical power provided by the exemplary system, method and computer-accessible medium according to an exemplary embodiment of the present disclosure, obtained by sending the modulated light through neutral density filters of different attenuation. The optical powers given can be corrected for modulation depth. The intersection of the asymptotes provided in the FIG. 8A can indicate the optical power corresponding to a signal to noise ratio of 1. (See FIG. 8B). A noise equivalent power of less than about 1.2 pW/VHz can be measured, which can be approaching the manufacturer specified noise floor of the APD module of about 0.8 pW/VHz.

FIG. 8A also illustrates an exemplary graph of responses of the neighboring, not driven, detectors. As can be seen in FIG. 8A, even at high optical input power into the main, driven, detector, the neighboring, non-driven, detectors show no signal. The channel separation on the detector side can be greater than about 100 DdB (20 log 10), and on the laser source side, there can be some measurable crosstalk between sources because of the proximity of the fibers in the optical multiplexer. A channel separation of greater than about 80 dB (20 log 10) can be measured.

The ADC converter can saturate when a signal of approximately 1.5 J1 W can be fed to the APD. This can be an order of magnitude higher than the performance of a commercial CW-NIRS system, and can also be much more than what can usually be seen in the transmission type measurements used in an exemplary breast scanner. Together with the noise floor of about 1.2 pW this can result in an instantaneous dynamic range of about 121 dB.

For example, neither inter-wavelength crosstalk nor amplitude to phase crosstalk can be observed in such exemplary situation. The phase noise of the output signal can be smaller than about 6 mrad/VHz at about 100 pW input power. Stability over about 10 hours was measured after leaving the instrument on for one hour in a climate controlled room. The measured amplitude changed by less than about 1.5%, and the phase less than about 3 mrad at an optical power of about 5 nW. The exemplary findings are summarized in Table 1 below.

TABLE 1
SUMMARY OF PERFORMANCE TESTS
TABLE 1 SUMMARY OF PERFORMANCE TESTS

| | |
|---|---|
| Noise floor | <1.2 pW/vHz |
| Phase Noise | <6 mrad/vHz @ 100 pW |
| Maximum Input Power | 1.5 μW |
| Dynamic Range | >110 dB (20log10) |
| Channel Separation | >80 dB (20log10) |
| Amplitude Stability | +−1.5% @ 10 hours, 5 nW |
| Phase Stability | 3 mrad @ 10 hours, 5 nW |

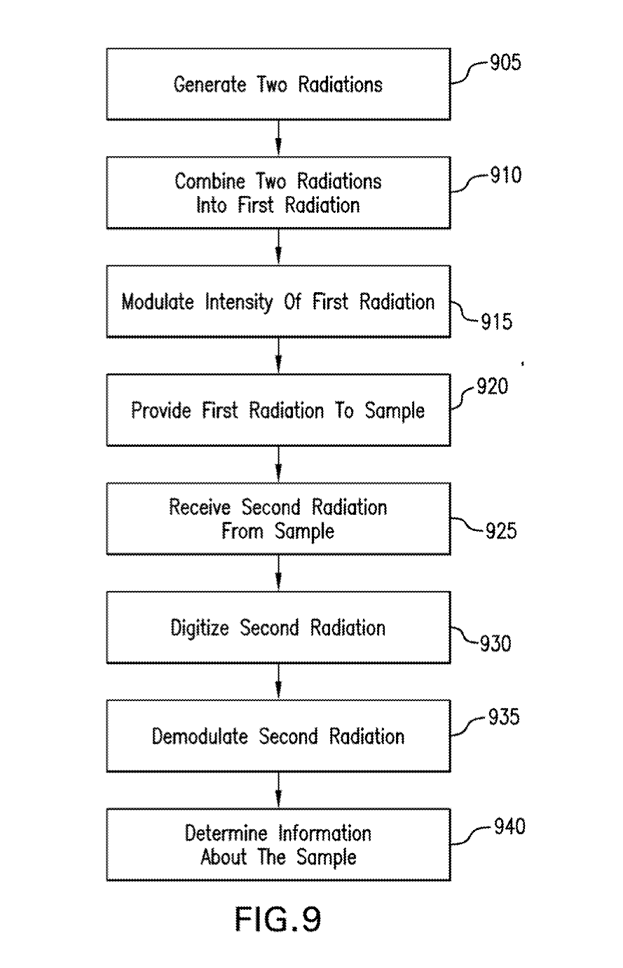

FIG. 9 illustrates a flow diagram of the method according to an exemplary embodiment of the present disclosure. In particular, at procedure 905, two radiations can be generated (e.g., through to laser arrangements). At procedure 910, the two radiations can be combined into a single first radiation (e.g., using a dichroic mirror). At procedure 915, the first radiation can be intensity modulated, and then provided to the sample at procedure 920. At procedure 925, the second radiation can be received from the sample, and it can be digitized at procedure 930. At procedure 935, the Second radiation can be demodulated, and the information about the sample can be determined at procedure 940.

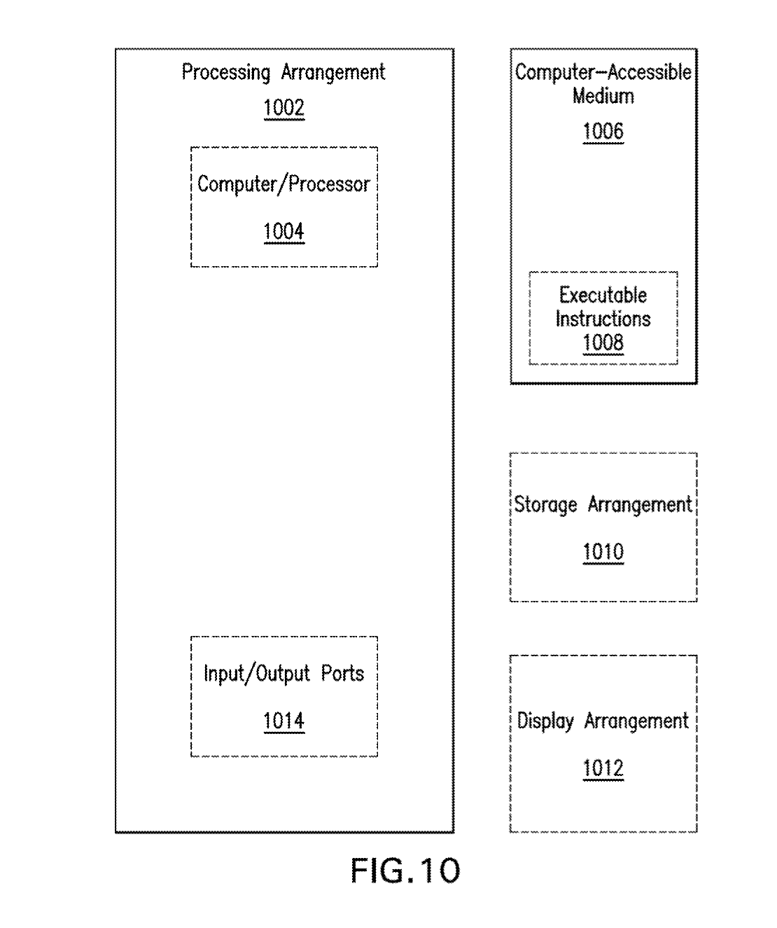

FIG. 10 shows a block diagram of an exemplary embodiment of a system according to the present disclosure. For example, exemplary procedures in accordance with the present disclosure described herein can be performed by a processing arrangement and/or a computing arrangement 1002. Such processing/computing arrangement 1002 can be, for example entirely or a part of, or include, but not limited to, a computer/processor 1004 that can include, for example one or more microprocessors, and use instructions stored on a computer-accessible medium (e.g., RAM, ROM, hard drive, or other storage device).

As shown in FIG. 10, for example a computer-accessible medium 1006 (e.g., as described herein above, a storage device such as a hard disk, floppy disk, memory stick, CD-ROM, RAM, ROM, etc., or a collection thereof) can be provided (e.g., in communication with the processing arrangement 1002). The computer-accessible medium 1006 can contain executable instructions 1008 thereon. In addition or alternatively, a storage arrangement 1010 can be provided separately from the computer-accessible medium 1006, which can provide the instructions to the processing arrangement 1002 so as to configure the processing arrangement to execute certain exemplary procedures, processes and methods, as described herein above, for example.

Further, the exemplary processing arrangement 1002 can be provided with or include an input/output arrangement 1014, which can include, for example a wired network, a wireless network, the internet, an intranet, a data collection probe, a sensor, etc. As shown in FIG. 10, the exemplary processing arrangement 1002 can be in communication with an exemplary display arrangement 1012, which, according to certain exemplary embodiments of the present disclosure, can be a touch-screen configured for inputting information to the processing arrangement in addition to outputting information from the processing arrangement, for example. Further, the exemplary display 1012 and/or a storage arrangement 1010 can be used to display and/or store data in a user-accessible format and/or user-readable format.

The foregoing merely illustrates the principles of the disclosure. Various modifications and alterations to the described embodiments will be apparent to those skilled in the art in view of the teachings herein. it will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. it should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entirety.
[1] Roche-labarbe, N., S, A. Carp, A. Surova, M. Patel, D. A. Boas, P. E. Grant and M. A. Franceschini {2010). “Noninvasive optical measures of CBV, StO(2), CBF Index, and rCMR0(2) in human premature neonates' brains in the first six weeks of life.” Hum Brain Mapp 31(3): 341-352.
[2] Martin Wolf, Marco Ferrari and Valentina Quaresima, “Progress of near-infrared spectroscopy and topography for brain and muscle clinical applications”, J. Biomed. Opt. 12, 062104 {Nov. 15, 2007); doi:10.1117/1.2804899
[3] Quan Zhang, Thomas J. Brukilacchio, Ang U, Jonathan J. Stott, Tina Chaves, Elizabeth Hillman, Tao Wu, MaryAnn Charlton, Elizabeth Rafferty, Richard H. Moore, Daniel B. Kopans and David A. Boas, “Coregistered tomographic X ray and optical breast imaging: initial results”, J. Biomed. Opt. 10, 024033 (Apr. 13, 2005); do1:10.1117/1.1899183
[4] Y. S. Yang, H. L. Uu, X. D. li, and B. Chance, “Low-cost frequency-domain photon migration instrument for tissue spectroscopy, oximetry, and Imaging,” Opt. Eng. 36, 1562-1569 {1997).
[5] B. Chance, M. Cope, E. Gratton, N. Ramanujam, and B. Tromberg, “Phase measurement of light absorption and scatter in human tissue”, Rev. Sci. Instrum. 69, 3457-3481 (1998).
[6] Fang, Q., S. A. Carp, J. Selb, G. Boverman, Q. Zhang, D. B. Kopans, R. H. Moore, E. l. Miller, D. H. Brooks and D. A. Boas (2009). “Combined optical imaging and mammography of the healthy breast: optical contrast derived from breast structure and compression.” IEEE Trans Med Imaging 28{1): 30-42.
[7] Carp, S. A., T. Kauffman, Q. Fang, E. Rafferty, R. Moore, D. Kopans and D. Boas (2006). “Compression-Induced changes in the physiological state of the breast as observed through frequency domain photon migration measurements.” J Blamed Opt 11{6): 064016.
[8] Carp, S. A., J. Selb, Q. Fang, R. Moore, D. B. Kopans, E. Rafferty and D. A. Boas (2008). “Dynamic functional and mechanical response of breast tissue to compression.” Opt Express 16(20):16064-16078.
[9] Oppenheim A V, Schafer R W: Discrete Time Signal Processing, Third Edition, ISBN-10: 0131988425 {Prentice Hall Press, Upper Saddle River, N.J., 2009)

1. An apparatus for determining information regarding at least one sample, comprising:
at least one source arrangement which provides at least one first radiation, whose intensity varies over time, to the at least one sample;
at least one detector arrangement which is configured to receive at least one second radiation from the at least one sample based on the at least one first radiation; and
at least one computer arrangement which is configured to simultaneously determine the information regarding the at least one sample at a plurality of frequencies of the at least one second radiation.

2. The apparatus according to claim 1, wherein at least one of the at least one first radiation or the at least one second radiation includes an optical radiation.

3. The apparatus according to claim 1, wherein the frequencies of the at least one second radiation are intensity modulated frequencies.

4. The apparatus according to claim 1, further comprising a frequency modulating arrangement which is configured to cause the at least one first radiation to be intensity modulated to provide a modulated first radiation to the at least one sample.

5. The apparatus according to claim 4, wherein the frequencies of the at least one second radiation are related to further frequencies of the intensity modulated first radiation.

6. The apparatus according to claim 1, wherein the at least one detector arrangement includes a demodulation arrangement which is configured to receive and simultaneously demodulate the at least one second radiation at the frequencies to generate demodulated information.

7. The apparatus according to claim 6, wherein the computer arrangement is further configured to determine the information regarding the at least one sample based on the demodulated information.

8. The apparatus according to claim 6, wherein the demodulation arrangement includes a digitizing arrangement which is configured to receive and digitize the at least one second radiation prior to the demodulation thereof.

9. The apparatus according to claim 6, wherein the demodulation arrangement includes at least one hardware adder circuitry which is configured to demodulate the at least one second radiation without a hardware multiplier arrangement.

10. The apparatus according to claim 8, further comprising a frequency modulating arrangement which is configured to cause the at least one first radiation to be intensity modulated to provide a modulated first radiation to the at least one sample, wherein the frequency modulating arrangement is further configured to modulate further frequencies of the at least one first radiation based on at least one sampling rate provided by the digitizing arrangement.

11. The apparatus according to claim 6, wherein the demodulation arrangement includes at least one hardware adder circuitry which is configured to provide modified information regarding the at least one second radiation, and wherein the computer arrangement is further configured to determine the information regarding the at least one sample based on the modified information.

12. The apparatus according to claim 11, wherein the computer arrangement is further configured to determine the information regarding the at least one sample based on the modified information to achieve a Discrete Fourier Transform (DFT) result of the at least one second radiation.

13. The apparatus according to claim 12, wherein the computer arrangement is further configured to individually compute at least two bins based on the at least one second radiation using the DFT and a Goertzel procedure.

14. The apparatus according to claim 1, wherein the at least on source arrangement includes at least two radiation generating arrangements configured to generate at least two further radiations, and further comprising a radiation combining arrangement configured to combine the at least two further radiations into at least one third radiation that is associated with the at least one first radiation.

15. The apparatus according to claim 14, wherein the radiation generating arrangements include lasers.

16. The apparatus according to claim 14, wherein the radiation combining arrangement includes a dichroic mirror.

17. The apparatus according to claim 14, further comprising at least one galvanometer configured to receive the at least one third radiation and generate at least one fourth radiation.

18. The apparatus according to claim 17, further comprising a microcontroller configured to adjust the galvanometer to a particular angle.

19. A method for determining information regarding at least one sample, comprising:
providing at least one first radiation, whose intensity varies over time, to the at least one sample;
receiving at least one second radiation from the at least one sample based on the at least one first radiation; and
using a computer hardware arrangement, simultaneously determining the information regarding the at least one sample at a plurality of frequencies of the at least one second radiation.

20-34. (canceled)

35. A non-transitory computer-accessible medium having stored thereon computer-executable instructions for determining information regarding at least one sample, wherein, when a computer arrangement executes the instructions, the computer arrangement is configured to perform procedures comprising:
causing an activation of at least one source arrangement which provides at least one first radiation, whose intensity varies over time, to the at least one sample;
receiving, using at least one detector arrangement, at least one second radiation from the at least one sample based on the at least one first radiation; and
simultaneously determining the information regarding the at least one sample at a plurality of frequencies of the at least one second radiation.

36-50. (canceled)
